(19) United States
(12) Patent Application Publication
(10) Pub. No.: US 2024/0249631 A1
(43) Pub. Date: Jul. 25, 2024

(54) METHOD AND APPARATUS FOR PROVIDING COGNITIVE-MOTOR TRAINING TO USER

(71) Applicant: AIBLE THERAPEUTICS CO., LTD., Seoul (KR)

(72) Inventors: Hyung Jun KIM; Yong Soo SHIM; Ji Hye PARK; Ho Sang CHEON; You Jin SHIN

(73) Assignee: AIBLE THERAPEUTICS CO., LTD., Seoul (KR)

(21) Appl. No.: 17/999,965

(22) PCT Filed: Nov. 2, 2022
(86) PCT No.: PCT/KR2022/017019

§ 371 Date: Nov. 28, 2022

(30) Foreign Application Priority Data
Dec. 8, 2021 (KR) 10-2021-0174797

(51) Int. Cl. G09B 5/02; G06T 7/70; G06V 40/10

(52) U.S. Cl. CPC G09B 5/02; G06T 7/70; G06V 40/10; G06T 2207/30196

(57) ABSTRACT

In order to provide cognitive-motor training to a user, an apparatus detects a user object in an image obtained by capturing the user, determines a target region in the user object, outputs content on the image, and performs an operation set for a target virtual object when the target region corresponds to the target virtual object among virtual objects.

### TECHNICAL FIELD

The technical field relates to technology for providing cognitive-motor training to a user, more particularly, to technology for providing cognitive-motor training to a user using an electronic device.

### Background Art

Dementia is one of the most serious diseases in life for the elderly. Especially in aging societies, the number of people diagnosed with dementia has been rapidly increasing over the last 10 years and the social and economic cost has been rapidly increasing as well. Dementia causes the patients and the family members who care for them great pain, since dementia makes a person unable to live independently and causes problems such as disappearance, suicide, and the like. If diagnosed early, dementia may be treated. Early diagnosis and appropriate treatment may prevent or delay additional decline in cognitive function. However, there are problems in the conventional technique for diagnosing dementia at an early stage. Conventionally, people who desire to have an examination need to visit a professional health care center, such as a hospital. However, many people do not visit a hospital until they notice they are becoming forgetful or their forgetfulness has worsened, by which time they have already developed mild cognitive impairment (MCI) or Alzheimer's disease (AD). Also, the reliability of a neuro-cognitive function test (e.g., Seoul neuropsychological screening battery-II (SNSB-II), Korean version of a consortium to establish a registry for Alzheimer's disease assessment packet (CERAD-K), etc.) to confirm a diagnosis can only be expected to be high when the test is conducted by experienced medical professionals with sufficient knowledge. In addition, examinations involving techniques, such as magnetic resonance imaging (MRI), single-photon emission computerized tomography (SPECT), positron emission tomography (PET), cerebrospinal fluid analysis, and the like, are expensive and being examined through such techniques make patients feel uncomfortable.

### DISCLOSURE OF THE INVENTION

### Technical Goals

An embodiment may provide a method and apparatus for providing cognitive-motor training to a user.

An embodiment may provide content used to provide cognitive-motor training to a user.

### Technical Solutions

According to an embodiment, a method of providing cognitive-motor training to a user, performed by an electronic device, may include detecting a user object in an image obtained by capturing a user, determining at least one preset target region in the object, outputting pre-produced content on the image, wherein the content includes one or more of virtual objects, determining whether the target region corresponds to a target virtual object among the virtual objects, and performing an operation set for the target virtual object when the target region corresponds to the target virtual object.

The determining of the at least one preset target region in the user object may include determining that at least one of a hand region and a foot region is the target region based on a human body model.

The determining of the at least one preset target region in the user object may include determining that at least one of a hand region and a foot region is the target region based on the user object through a preset application programming interface (API).

At least one of the one or more of the virtual objects may be different from another object in at least one of shape, color, and position.

The pre-produced content may include a text or a voice with which the user is able to identify the target virtual object.

The determining of whether the target region corresponds to the target virtual object among the virtual objects may include determining whether the target region at least partially overlaps the target virtual object in the image, and determining that the target region corresponds to the target virtual object when the target region at least partially overlaps the target virtual object.

The determining of whether the target region corresponds to the target virtual object among the virtual objects may further include determining whether a pose of the user object is a target pose, and the determining that the target region corresponds to the target virtual object when the target region at least partially overlaps the target virtual object may include determining that the target region corresponds to the target virtual object when the target region overlaps the target virtual object and the pose of the user object is the target pose.

The method may further include outputting a result of analyzing the content based on a result of a user's performance related to the content.

According to an embodiment, an electronic device for providing cognitive-motor training to a user may include a memory configured to store a program for providing cognitive-motor training to a user and a processor configured to perform the program, wherein the program may include detecting a user object in an image obtained by capturing a user, determining at least one preset target region in the user object, outputting pre-produced content on the image, wherein the content includes one or more of virtual objects, determining whether the target region corresponds to a target virtual object among the virtual objects, and performing an operation set for the target virtual object when the target region corresponds to the target virtual object.

The determining of the at least one preset target region in the user object may include determining that at least one of a hand region and a foot region is the target region based on a human body model.

The determining of the at least one preset target region in the user object may include determining that at least one of a hand region and a food region is the target region based on the user object through a predefined API.

At least one of the one or more of the virtual objects may be different from another object in at least one of shape, color, and position.

The pre-produced content may include a text or a voice with which the user is able to identify the target virtual object.

The determining of whether the target region corresponds to the target virtual object among the virtual objects may include determining whether the target region at least partially overlaps the target virtual object in the image, and determining that the target region corresponds to the target virtual object when the target region at least partially overlaps the target virtual object.

The determining of whether the target region corresponds to the target virtual object among the virtual objects may further include determining whether a pose of the user object is a target pose, and the determining that the target region corresponds to the target virtual object when the target region at least partially overlaps the target virtual object may include determining that the target region corresponds to the target virtual object when the target region corresponds to the target virtual object and the pose of the user object is the target pose.

The program may be further configured to perform outputting a result of analyzing the content based on a result of a user's performance related to the content.

### Effects

A method and apparatus for providing cognitive-motor training to a user may be provided.

Content used to provide cognitive-motor training to a user may be provided.

### BRIEF DESCRIPTION OF DRAWINGS

FIG. 1 is a diagram illustrating a configuration of a system for providing cognitive-motor training to a user according to an embodiment.

FIG. 2 is a diagram illustrating a configuration of an electronic device for providing cognitive-motor training to a user according to an embodiment.

FIG. 3 is a flowchart illustrating a method of providing cognitive-motor training to a user according to an embodiment.

FIG. 4 is a diagram illustrating target regions in a user object according to an embodiment.

FIG. 5 is a flowchart illustrating a method of determining whether a target region corresponds to a target virtual object among virtual objects according to an embodiment.

FIG. 6 is a diagram illustrating pre-produced content according to an embodiment.

FIG. 7 is a diagram illustrating pre-produced content according to another embodiment.

FIG. 8 is a diagram illustrating pre-produced content according to yet another embodiment.

FIG. 9 is a diagram illustrating pre-produced content according to still another embodiment.

FIG. 10 is a diagram illustrating an interaction with a user through content according to an embodiment.

FIG. 11 is a flowchart illustrating a method of outputting a result of analyzing content based on a result of a user's performance according to an embodiment.

### BEST MODE FOR CARRYING OUT THE INVENTION

Hereinafter, embodiments will be described in detail with reference to the accompanying drawings. The scope of the right, however, should not be construed as limited by the embodiments set forth herein. In the drawings, like reference numerals are used for like elements.

Various modifications may be made to the embodiments. Here, the embodiments are not to be construed as limited by the disclosure and should be construed as including all changes, equivalents, and replacements within the idea and the technical scope of the disclosure.

The terminology used herein is for the purpose of describing particular embodiments only and is not intended to limit the embodiments. The singular forms “a”, “an”, and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms including technical and scientific terms used herein have the same meaning as commonly understood by one of ordinary skill in the art to which the embodiments belong. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

When describing the embodiments with reference to the accompanying drawings, like reference numerals refer to like constituent elements and any repeated description related thereto will be omitted. In the description of any embodiment, any detailed description of well-known related structures or functions will be omitted when it is deemed that such description will make the present disclosure ambiguous and open to different interpretations.

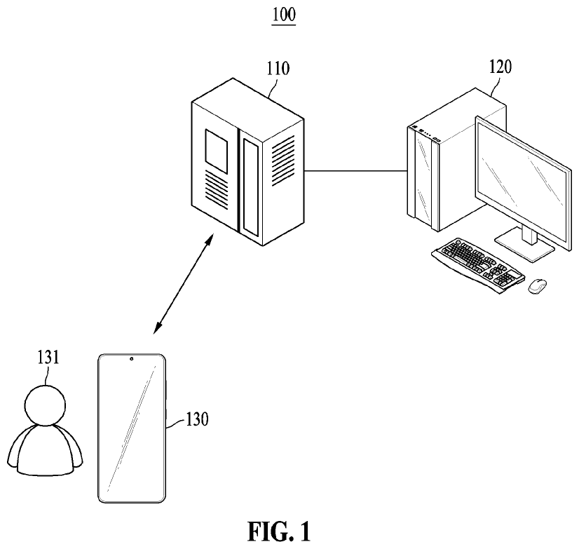

FIG. 1 is a diagram illustrating a configuration of a system for providing cognitive-motor training to a user according to an embodiment.

According to an embodiment, a system 100 for providing cognitive-motor training to a user may include a service server 110, an administrator terminal 120 that may access data of the service server 110, and a user terminal 130. For example, a user 131 may receive cognitive-motor training through his or her user terminal 130. Cognitive-motor training may be to train the user 131 to think about a current situation through a mental or conscious process and move his or her body accordingly. When the user 131 performs cognitive-motor training, cognitive stimulation and motor stimulation may be performed together.

A program, software, or an application including contents produced to provide cognitive-motor training to the user 131 may be installed on the user terminal 130 in advance. The user terminal 130 may generate result information on a result of the user 131 using the contents and transmit the generated result information to the server 110. The server 110 may generate a result of analyzing the result information based on the result information and store the analysis result in the server 110 or provide the analysis result to the user 131 through the user terminal 130.

The user terminal 130 may be a mobile terminal such as a tablet and a smartphone. If the user terminal 130 is a mobile terminal, the user 131 may perform cognitive-motor training without being restricted by time and space.

An administrator of the system 100 or a user (e.g., a medical professional) of the administrator terminal 120 that is allowed access to personal information of the user 131 may monitor a training result of the user 131.

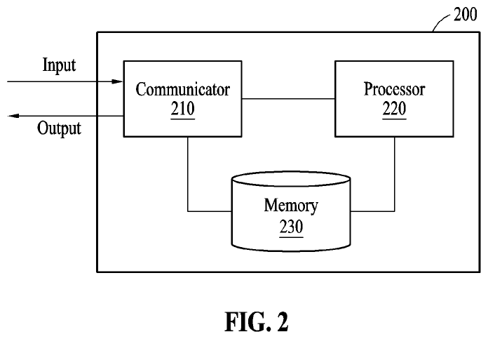

A method of providing cognitive-motor training to a user is described in detail below with reference to FIGS. 2 through 11.

FIG. 2 is a diagram illustrating a configuration of an electronic device for providing cognitive-motor training to a user according to an embodiment.

An electronic device 200 may include a communicator 210, a processor 220, and a memory 230. For example, the electronic device 200 may be the user terminal 130 described above with reference to FIG. 1.

The communicator 210 is connected to the processor 220 and the memory 230 and transmits and receives data to and from the processor 220 and the memory 230. The communicator 210 may be connected to another external apparatus and transmit and receive data to and from the external apparatus. Hereinafter, transmitting and receiving “A” may refer to transmitting and receiving “information or data indicating A”.

The communicator 210 may be implemented as circuitry in the electronic device 200. For example, the communicator 210 may include an internal bus and an external bus. As another example, the communicator 210 may be an element that connects the electronic device 200 to the external apparatus. The communicator 210 may be an interface. The communicator 210 may receive data from the external apparatus and transmit the data to the processor 220 and the memory 230.

The processor 220 may process data received by the communicator 210 and stored in the memory 230. A “processor” described herein may be a hardware-implemented data processing apparatus having a physically structured circuit to execute desired operations. For example, the desired operations may include code or instructions included in a program. For example, the hardware-implemented data processing apparatus may include a microprocessor, a central processing unit (CPU), a processor core, a multi-core processor, a multiprocessor, an application-specific integrated circuit (ASIC), and a field-programmable gate array (FPGA).

The processor 220 executes a computer-readable code (for example, software) stored in a memory (for example, the memory 230) and instructions triggered by the processor 220.

The memory 230 stores the data received by the communicator 210 and data processed by the processor 220. For example, the memory 230 may store the program (or an application, or software). The stored program may be a set of syntaxes that are coded and executable by the processor 220 to provide cognitive-motor training to a user.

The memory 230 may include, for example, at least one volatile memory, nonvolatile memory, random-access memory (RAM), flash memory, hard disk drive, and optical disc drive.

The memory 230 may store an instruction set (for example, software) for operating the electronic device 200. The instruction set for operating the electronic device 200 is executed by the processor 220.

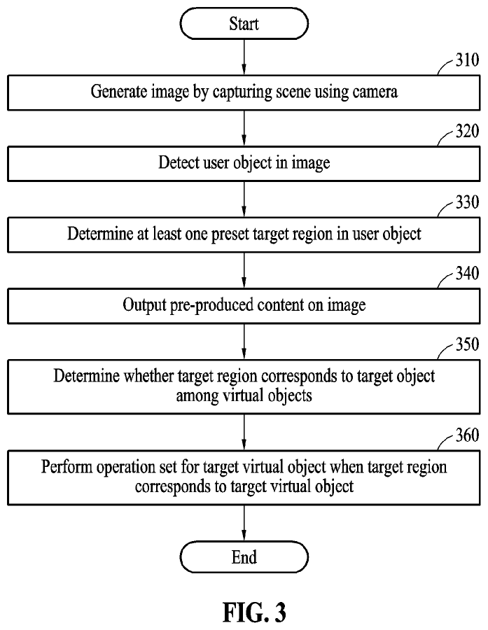

The communicator 210, the processor 220, and the memory 230 are described in detail below with reference to FIGS. 3 through 11.

FIG. 3 is a flowchart illustrating a method of providing cognitive-motor training to a user according to an embodiment.

Operations 310 through 360 described below are performed by the electronic device 200 described above with reference to FIG. 2.

In operation 310, the electronic device 200 may generate an image by capturing a scene using a camera. The camera used to generate the image may generally be a camera embedded in the electronic device 200, but examples are not limited thereto. For example, the electronic device 200 may generate the image using the camera connected to the electronic device 200 by wire or wirelessly.

In operation 320, the electronic device 200 may detect a user object in the image. For example, the user object may be an object corresponding to a shape of a user (e.g., the user 131 of FIG. 1) of the electronic device 200. Various techniques may be used to detect a user object in an image, and a technique is not limited to a specific technique. For example, the electronic device 200 may use a first application programming interface (API) that is preset to detect the user object. The electronic device 200 may track a movement of the user object through continuously generated images.

In operation 330, the electronic device 200 may determine at least one preset target region in the user object. For example, the target region may include a human head, left hand, right hand, left foot, right foot, left knee, right knee, and the like, but examples are not limited thereto. As another example, the electronic device 200 may determine a pose of the user object and determine the target region based on the pose.

According to an embodiment, the electronic device 200 may track a movement of the target region of the user object through the continuously generated images. The electronic device 200 may use a second API that is preset to detect and track the target region in the user object. The second API may be identical to the first API used to detect the user object, and the second API and the first API may be different depending on an embodiment.

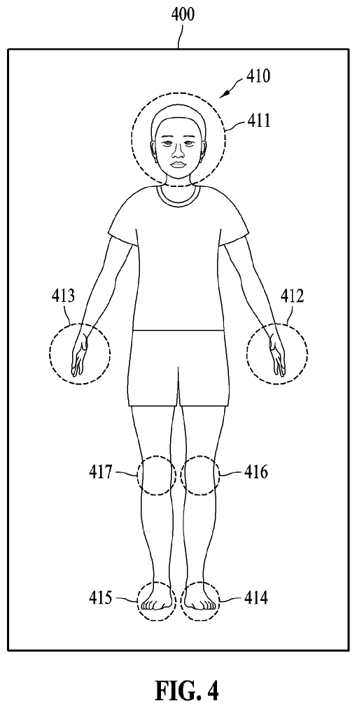

For example, the electronic device 200 may determine that at least one of a hand region and a foot region is the target region and track the target region based on the user object through the second API. The target region determined based on the user object is described in detail below with reference to FIG. 4.

In operation 340, the electronic device 200 may output pre-produced content on the image.

According to an embodiment, the content may include one or more of virtual objects. For example, the virtual objects may be computer graphic (CG) objects for augmented reality (AR). In addition, the content may further include an instruction for the user to interact with the virtual objects. For example, the instruction may be a text or voice instruction describing a target virtual object among the virtual objects and describing how to interact with the target virtual object. The instruction may include a text or a voice with which the user may identify the target virtual object.

According to an embodiment, when multi-color balloons are output as the virtual objects on the image, the instruction may be “Touch a red balloon with your right hand.”

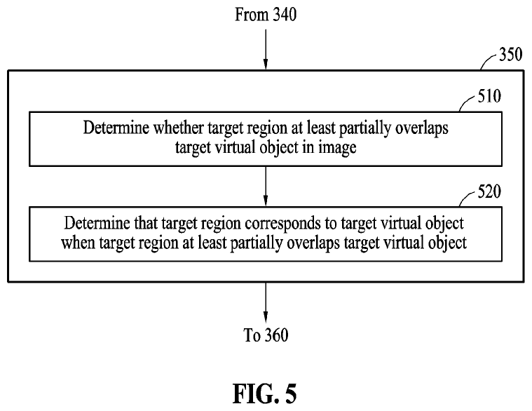

A plurality of pre-produced contents may be provided, and each of the contents may be provided in a form of a step or a stage to the user. Each of the contents may be produced for predetermined cognitive-motor training. The contents are described in detail with reference to FIGS. 5 through 8.

The user may move his or her body in response to the instruction that is output. For example, if the instruction is “Touch the red balloon with your right hand,” the user may recognize the red balloon among one or more of balloons and move his or her right hand such that his or her right hand corresponds to a position of the red balloon.

In operation 350, the electronic device 200 may determine whether the target region that is being tracked corresponds to the target virtual object among the virtual objects. In the above-described embodiment, it may be determined whether the right hand, which is the target region of the user, corresponds to the red balloon, which is the target virtual object. For example, when the target region at least partially overlaps the target virtual object in the image, it may be determined that the target region corresponds to the target virtual object.

In operation 360, when the target region corresponds to the target virtual object, the electronic device 200 may perform an operation set for the target virtual object. For example, if the virtual object is a balloon, a visual effect of a balloon popping may appear. As another example, if the virtual object is a balloon, a sound effect of a balloon popping may appear. As yet another example, the operation set for the target virtual object may be to increase a score. Various operations may be set for the target virtual object, and examples are not limited to the above-described embodiment.

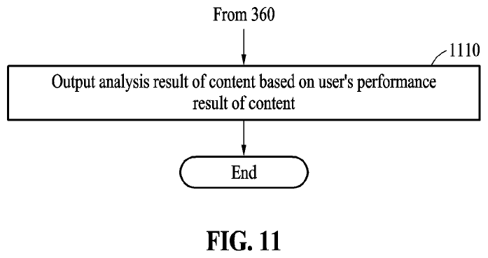

According to the above-described embodiment, the user may recognize (or identify) a designated target virtual object among the virtual objects output through the content and move his or her body by performing an action designated for the target virtual object. Since the user performs cognitive function and motor function through the content, it may be evaluated that a purpose of producing the content is achieved. Additionally, the user may be evaluated based on a result of his or her performance related to the content. Hereinafter, a technique of generating a result of an analysis based on the result of the user's performance related to the content is described in detail with reference to FIG. 11.

FIG. 4 is a diagram illustrating target regions in a user object according to an embodiment.

According to an embodiment, the electronic device 200 may detect a user object 410 in an image 400 captured by a camera. For example, the electronic device 200 may detect the user object 410 in the image 400 using a first API.

The electronic device 200 may determine at least one preset target region based on the detected user object 410. For example, the electronic device 200 may detect and track the user object 410 in the image 400 using a second API (or the first API). For example, target regions may include a human head 411, left hand 412, right hand 413, left foot 414, right foot 415, left knee 416, right knee 417, and the like, but examples are not limited thereto.

According to an embodiment, the electronic device 200 may generate a human body model of the user based on the user object 410, and the target regions may be determined based on the human body model.

According to an embodiment, virtual objects of content may be output on the image 400 to overlap each other.

FIG. 5 is a flowchart illustrating a method of determining whether a target region corresponds to a target virtual object among virtual objects according to an embodiment.

According to an embodiment, operation 350 described above with reference to FIG. 3 may include operations 510 and 520 described below.

In operation 510, the electronic device 200 may determine whether a target region at least partially overlaps a target virtual object in an image. For example, if an instruction from content is “Touch a red balloon with your right hand,” it may be determined whether a target region corresponding to a right hand of a user overlaps the red balloon.

In operation 520, when the target region at least partially overlaps the target virtual object, the electronic device 200 may determine that the target region corresponds to the target virtual object. When the target region corresponds to the target virtual object, it may be evaluated that the user properly followed the instruction.

According to an embodiment, the electronic device 200 may calculate that a time from when an instruction is given to the user or when the virtual objects are output until it is determined that the target region corresponds to the target virtual object is a performance time. The performance time may be included later, in a performance result related to the content.

According to an embodiment, the electronic device 200 may determine whether a pose of a user object is a target pose. For example, the electronic device 200 may determine whether the pose of the user object is the target pose based on a human body model of the user. For example, the target pose may be transmitted to the user through the instruction from the content.

When the target region overlaps the target virtual object and the pose of the user object is the target pose, the electronic device 200 may determine that the target region corresponds to the target virtual object.

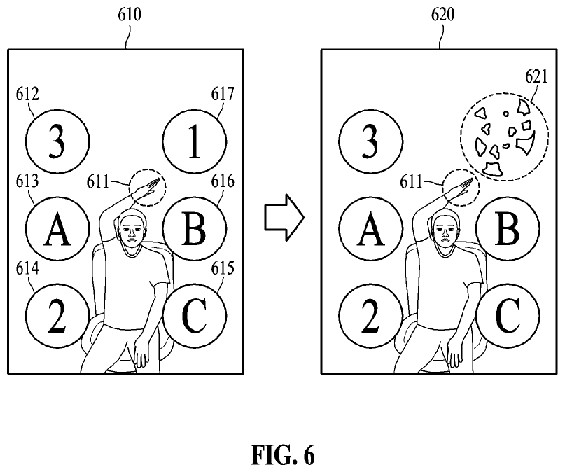

FIG. 6 is a diagram illustrating pre-produced content according to an embodiment.

According to an embodiment, content for a warm-up may be produced in advance. The content may output virtual objects 612 through 617 on an image 610 including a user object. At least one of the virtual objects 612 through 617 may be different from another object in at least one of shape, color, and position. For example, a first instruction output to a user may be “Touch ‘1’ with your right hand.”

The electronic device 200 may determine whether a right hand 611 of the user as a target region overlaps a virtual object 617 as a target virtual object among the virtual objects 612 through 617 and perform an operation set for the virtual object 617 when the right hand 611 overlaps the virtual object 617. For example, the operation set for the virtual object 617 may be an effect of the virtual object 617 popping. A virtual object 621 of a balloon popping may be output on an image 620 instead of the virtual object 617. As another example, when the user touches the virtual object 617 with his or her left hand instead of his or her right hand or when the user touches another virtual object (e.g., a virtual object 614) with his or her right hand, a corresponding virtual object may not pop, and an effect of the virtual object being pushed from and returning to its original position may be output.

When the first instruction is properly performed, a second instruction may be performed subsequently. For example, the second instruction may be “Touch ‘A’ with your right hand.” A third instruction “Touch ‘2’ with your right hand” may be output subsequently.

According to an embodiment, the user may understand instruction regularity through the instructions and predict (or infer) an instruction to be output next based on the regularity. Through such process, cognitive ability of the user may be trained.

According to an embodiment, the electronic device 200 may generate a performance result related to the content. For example, the performance result may include a performance time. As another example, the performance result may include a number of mistakes made by the user when following instructions.

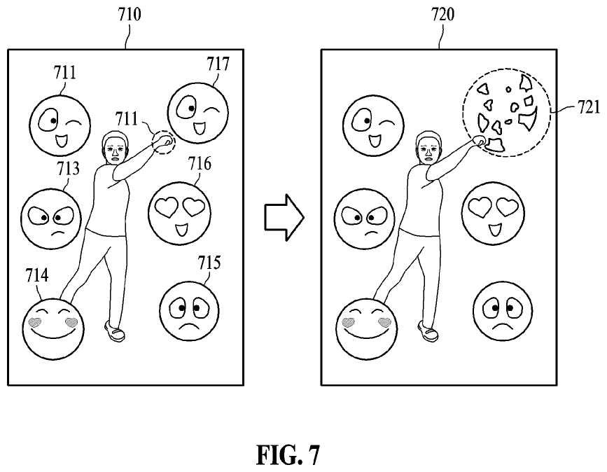

FIG. 7 is a diagram illustrating pre-produced content according to another embodiment.

According to an embodiment, content for a cardio workout may be produced in advance. The content may output virtual objects 712 through 717 on an image 710 including a user object. At least one of the virtual objects 712 through 717 may be different from another virtual object in at least one of shape, color, and position. For example, an instruction output to a user may be “Touch identical pictures alternately with your right and left fist.”

The electronic device 200 may determine whether a right hand 711 of the user as a target region overlaps a virtual object 717 as a target virtual object among the virtual objects 712 through 717 and perform an operation set for the virtual object 717 when the right hand 711 overlaps the virtual object 717. For example, the operation set for the virtual object 717 may be an effect of the virtual object 717 popping. A virtual object 721 of a balloon popping may be output on an image 720 instead of the virtual object 717. Subsequently, the user may touch the virtual object 721, which is a picture identical to the virtual object 717, with a hand opposite to the right hand 711, that is, with his or her left hand.

According to an embodiment, the user may train short-term memory by memorizing meanings and positions of pictures shown through the instruction. Through such process, cognitive ability and cardiovascular endurance of the user may be trained.

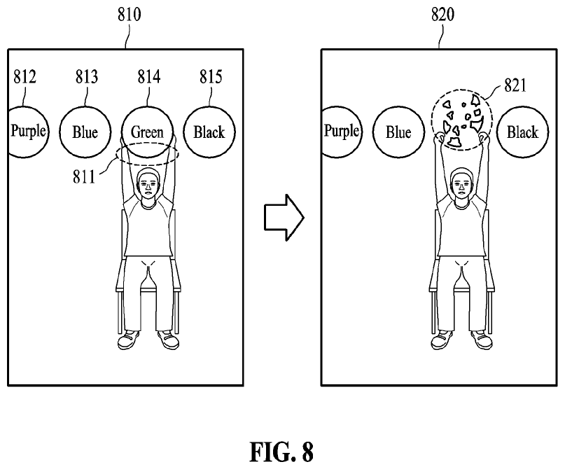

FIG. 8 is a diagram illustrating pre-produced content according to yet another embodiment.

According to an embodiment, content for a strength workout may be produced in advance. The content may be virtual objects 812 through 815 output on an image 810 including a user object. At least one of the virtual objects 812 through 815 may be different from another virtual object in at least one of shape, color, and position. For example, an instruction output to a user may be “With both hands touch a picture including a word that is a name of a color and the color name matches an ink color of the word.” Positions of the virtual objects 812 through 815 may move in one direction (e.g., from left to right) in the image 810. For example, a virtual object 814 among the illustrated virtual objects 812 through 815 may include a word that is a name of a color and the color name matches an ink color of the word, and other virtual objects 811, 812, 813, and 815 may each include a word that is a name of a color and the color name does not match an ink color of the word.

The user may touch the picture by raising both hands above his or her head. For example, the user may enhance an effect of the strength workout by performing an action of lifting an object with a little weight.

The electronic device 200 may determine whether both hands 811 of the user as a target region overlap the virtual object 814 as a target virtual object among the virtual objects 812 through 815 and perform an operation set for the virtual object 814 when both hands 811 overlap the virtual object 814. For example, the operation set for the virtual object 814 may be an effect of the virtual object 814 popping. A virtual object 821 of a balloon popping may be output on an image 820 instead of the virtual object 814.

According to an embodiment, the user may use the content to train concentration and body control. Through such process, cognitive ability and muscular strength of the user may be trained.

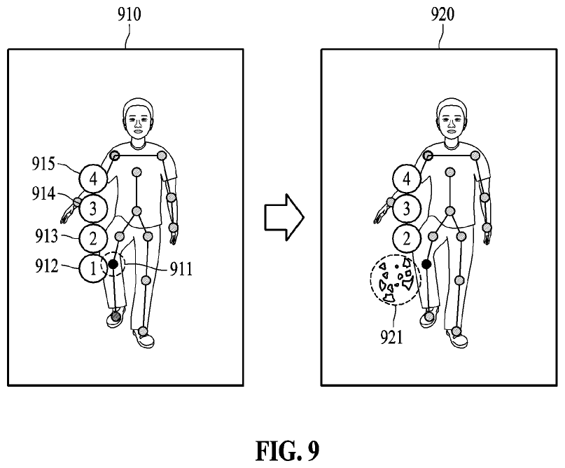

FIG. 9 is a diagram illustrating pre-produced content according to still another embodiment.

According to an embodiment, content for a cool down workout may be produced in advance. The content may be virtual objects 912 through 915 output on an image 910 including a user object. At least one of the virtual objects 912 through 915 may be different from another virtual object in at least one of shape, color, and position.

For example, an instruction output to a user may be “Lift your right knee and touch pictures in a numbered order with your knee.” Positions of the virtual objects 912 through 915 may be output near a position of a body part to be exercised. For example, if the body part is a right knee, the virtual objects 912 through 915 may be output near a position of the right knee.

The user may lift his or her right knee and touch the virtual objects 912 through 915 one at a time.

The electronic device 200 may determine whether a right knee 911 of the user as a target region overlaps a virtual object 912 as a target virtual object among the virtual objects 912 through 915 and perform an operation set for the virtual object 912 when the right knee 911 overlaps the virtual object 912. For example, the operation set for the virtual object 912 may be an effect of the virtual object 912 popping. A virtual object 921 of a balloon popping may be output on an image 920 instead of the virtual object 912. Subsequently, the user may touch a virtual object 913 by lifting his or her right knee again.

According to an embodiment, the positions of the objects 912 through 915 may guide the user to workout properly.

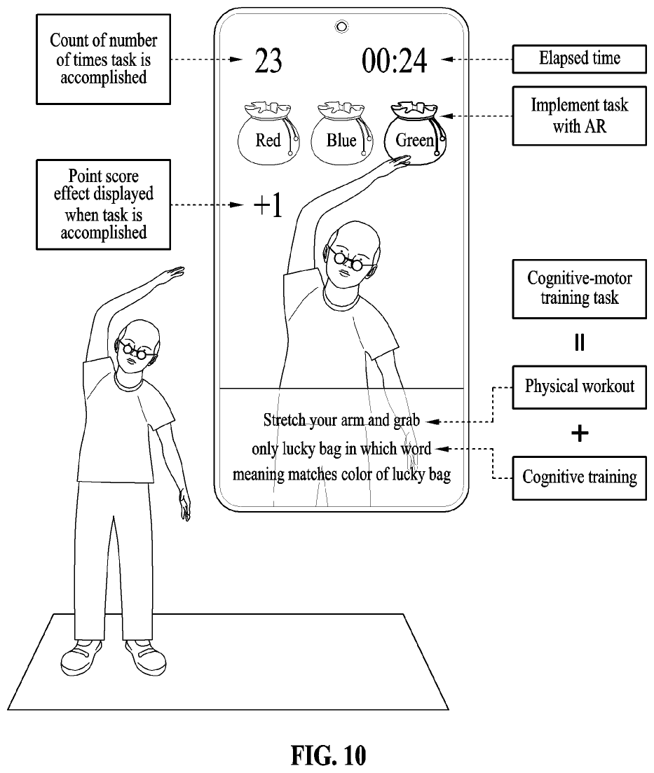

FIG. 10 is a diagram illustrating an interaction with a user through content according to an embodiment.

According to an embodiment, content may provide the user with cognitive training and a physical workout at the same time. For example, the content may be produced in a form of a quest. In order to give the user a great sense of achievement that comes with completing a quest, the content may provide feedback by outputting a visual effect or a sound effect when the user completes the quest and may quantify a performance result related to the content. Examples of the quantification of the feedback and performance result are not limited to the illustrated embodiment.

The content may stimulate and activate multiple brain areas of the user by providing dual tasks to enable the user to perform cognitive training and a physical workout at the same time instead of providing a single task. In addition, since the content is produced using gamification, it may be possible to increase an immersive experience of the user and encourage the user to participate voluntarily and continuously.

FIG. 11 is a flowchart illustrating a method of outputting a result of analyzing content based on a result of a user's performance according to an embodiment.

According to an embodiment, operation 1110 may be further performed after operation 360 described above with reference to FIG. 3 is performed.

In operation 1110, the electronic device 200 may output a result of analyzing content based on a result of a user's performance related to the content.

According to an embodiment, the electronic device 200 may output actual performance compared to a goal set for the content based on the performance result related to the content. The analysis result may include a level of a user to be evaluated based on demographic information (e.g., age, gender, height, weight, etc.) about the user.

According to an embodiment, the electronic device 200 may transmit the performance result related to the content to a server (e.g., the server 110 of FIG. 1) connected to the electronic device and receive a result of analyzing the performance result from the server. The server 110 may calculate preset various indices based on the performance result and include the calculated indices in the analysis result. For example, the indices may include a degree of dementia. The server 110 may check progress of dementia a user may experience by monitoring the user's performance result and the analysis result over a long period of time. When the degree of dementia the user experiences corresponds to a preset degree of dementia, the server 110 may suggest to the user that he or she see an expert, such as a doctor, and have an examination.

According to an embodiment, the expert, such as a doctor, who takes care of the user, may monitor a state of the user using the administrator terminal 120.

The units described herein may be implemented using a hardware component, a software component and/or a combination thereof. A processing apparatus may be implemented using one or more general-purpose or special purpose computers, such as, for example, a processor, a controller and an arithmetic logic unit (ALU), a DSP, a microcomputer, an FPGA, a programmable logic unit (PLU), a microprocessor or any other apparatus capable of responding to and executing instructions in a defined manner. The processing apparatus may run an operating system (OS) and one or more software applications that run on the OS. The processing apparatus also may access, store, manipulate, process, and create data in response to execution of the software. For purpose of simplicity, the description of a processing apparatus is used as singular; however, one skilled in the art will appreciate that a processing apparatus may include multiple processing elements and multiple types of processing elements. For example, a processing apparatus may include multiple processors or a processor and a controller. In addition, different processing configurations are possible, such as parallel processors.

The software may include a computer program, a piece of code, an instruction, or some combination thereof, to independently or uniformly instruct or configure the processing apparatus to operate as desired. Software and data may be embodied permanently or temporarily in any type of machine, component, physical or virtual equipment, computer storage medium or apparatus, or in a propagated signal wave capable of providing instructions or data to or being interpreted by the processing apparatus. The software also may be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD-ROM discs or DVDs; magneto-optical media such as optical discs; and hardware apparatuses that are specially configured to store and perform program instructions, such as read-only memory (ROM), RAM, flash memory, and the like. Examples of program instructions include both machine code, such as produced by a compiler, and files containing higher-level code that may be executed by the computer using an interpreter. The above-described apparatuses may be configured to act as one or more software modules in order to perform the operations of the above-described example examples, or vice versa.

A number of embodiments have been described above. Nevertheless, it should be understood that various modifications may be made to these embodiments. Suitable results may be achieved if the described techniques are performed in a different order, and/or if components in a described system, architecture, apparatus, or circuit are combined in a different manner, and/or replaced or supplemented by other components or their equivalents.

Therefore, the scope of the disclosure is defined not by the detailed description, but by the claims and their equivalents, and all variations within the scope of the claims and their equivalents are to be construed as being included in the disclosure.

1. A method of providing cognitive-motor training to a user, performed by an electronic device, the method comprising:
detecting a user object in an image obtained by capturing a user;
determining at least one preset target region in the user object;
outputting pre-produced content on the image, wherein the content comprises one or more of virtual objects;
determining whether the target region corresponds to a target virtual object among the virtual objects; and
performing an operation set for the target virtual object when the target region corresponds to the target virtual object.

2. The method of claim 1, wherein the determining of the at least one preset target region in the user object comprises determining that at least one of a hand region and a foot region is the target region based on a human body model.

3. The method of claim 1, wherein the determining of the at least one preset target region in the user object comprises determining that at least one of a hand region and a foot region is the target region based on the user object through a preset application programming interface (API).

4. The method of claim 1, wherein at least one of the one or more of the virtual objects is different from another object in at least one of shape, color, and position.

5. The method of claim 1, wherein the pre-produced content comprises a text or a voice with which the user is able to identify the target virtual object.

6. The method of claim 1, wherein the determining of whether the target region corresponds to the target virtual object among the virtual objects comprises:
determining whether the target region at least partially overlaps the target virtual object in the image; and
determining that the target region corresponds to the target virtual object when the target region at least partially overlaps the target virtual object.

7. The method of claim 6, wherein
the determining of whether the target region corresponds to the target virtual object among the virtual objects further comprises determining whether a pose of the user object is a target pose, and
the determining that the target region corresponds to the target virtual object when the target region at least partially overlaps the target virtual object comprises determining that the target region corresponds to the target virtual object when the target region overlaps the target virtual object and the pose of the user object is the target pose.

8. The method of claim 1, further comprising:
outputting a result of analyzing the content based on a result of a user's performance related to the content.

9. A non-transitory computer-readable storage medium storing instructions that, when executed by a processor, cause the processor to perform the method of claim 1.

10. An electronic device for providing cognitive-motor training to a user, the electronic device comprising:
a memory configured to store a program for providing cognitive-motor training to a user; and
a processor configured to perform the program,
wherein the program comprises:
detecting a user object in an image obtained by capturing a user;
determining at least one preset target region in the user object;
outputting pre-produced content on the image, wherein the content comprises one or more of virtual objects;
determining whether the target region corresponds to a target virtual object among the virtual objects; and
performing an operation set for the target virtual object when the target region corresponds to the target virtual object.

11. The electronic device of claim 10, wherein the determining of the at least one preset target region in the user object comprises determining that at least one of a hand region and a foot region is the target region based on a human body model.

12. The electronic device of claim 10, wherein the determining of the at least one preset target region in the user object comprises determining that at least one of a hand region and a foot region is the target region based on the user object through a predefined application programming interface (API).

13. The electronic device of claim 10, wherein at least one of the one or more of the virtual objects is different from another object in at least one of shape, color, and position.

14. The electronic device of claim 10, wherein the pre-produced content comprises a text or a voice with which the user is able to identify the target virtual object.

15. The electronic device of claim 10, wherein the determining of whether the target region corresponds to the target virtual object among the virtual objects further comprises:
determining whether the target region at least partially overlaps the target virtual object in the image; and
determining that the target region corresponds to the target virtual object when the target region at least partially overlaps the target virtual object.

16. The electronic device of claim 15, wherein
the determining of whether the target region corresponds to the target virtual object among the virtual objects further comprises determining whether a pose of the user object is a target pose, and
the determining that the target region corresponds to the target virtual object when the target region at least partially overlaps the target virtual object comprises determining that the target region corresponds to the target virtual object when the target region corresponds to the target virtual object and the pose of the user object is the target pose.

17. The electronic device of claim 10, wherein the program is further configured to perform outputting a result of analyzing the content based on a result of a user's performance related to the content.
